SEQUENCE LISTING (2 sequences)

SEQ ID NO: 1 - skipped

SEQ ID NO: 2 - DNA, 25 bp, synthetic construct
  source 1..25: mol_type = other DNA
aaacaatggagtcctttacaaactc

(19) United States
(12) Patent Application Publication
(10) Pub. No.: US 2025/0099449 A1
(43) Pub. Date: Mar. 27, 2025

(54) Methods for Sensitizing Gliomas to Immunotherapy

(71) Applicant: Northwestern University, Evanston, IL (US)

(72) Inventors: Joseph Thomas Duffy, Evanston, IL (US); Irina V. Balyasnikova, Evanston, IL (US)

(21) Appl. No.: 18/776,064

(22) Filed: Jul. 17, 2024

Related U.S. Application Data
(60) Provisional application No. 63/514,035, filed on Jul. 17, 2023.

(51) Int. Cl. A61K 31/444; A61K 45/06; A61P 43/00

(52) U.S. Cl. CPC A61K 31/444; A61K 45/06; A61P 43/00

(57) ABSTRACT

The present invention provides methods and compositions for sensitizing glioma cells to immunotherapy by contacting the cells with a Sec61 complex inhibitor.

### CROSS-REFERENCE TO RELATED APPLICATIONS

This application claims the benefit of and priority to U.S. Provisional Application No. 63/514,035 filed on Jul. 17, 2023. The content of which is incorporated by reference in its entirety.

### STATEMENT REGARDING FEDERALLY SPONSORED RESEARCH

N/A

### REFERENCE TO SEQUENCE LISTING

The contents of the electronic sequence listing (702581.02541.xml; Size: 1,999 bytes; and Date of Creation: Jul. 17, 2024) is herein incorporated by reference in its entirety.

### BACKGROUND

Glioblastoma is an incurable brain tumor. The current standard of care treatment, surgical resection, radiation, and temozolomide therapy, extends the survival of patients by only about 2.5 months. Despite demonstrating promise and success in select peripheral cancers, immunotherapy in GBM has yet to show clinical benefit. Several barriers exist in GBM that hinder the immunotherapeutic response. Therefore, improved treatments for glioblastoma are needed.

### SUMMARY

In an aspect, provided herein is a method for sensitizing glioma cells to immunotherapy in a subject in need thereof, the method comprising administering to the subject an inhibitor of the Sec61 complex. The inhibitor may reduce at least one of the expression of a Sec61 complex subunit and the activity of a Sec61 complex subunit. The Sec61 complex subunit may be Sec61G.

In an embodiment, the inhibitor comprises a small-molecular therapeutic agent. The inhibitor may be

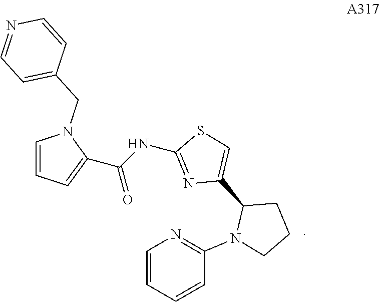 [SMILES: O=C(Nc1nc([C@H]2CCCN2c2ccccn2)cs1)c1cccn1Cc1ccncc1]

In an embodiment, the inhibitor comprises a genome editing system.

The method may further comprise administering to the subject an immunotherapy for the glioma cells. In an embodiment, the Sec61 complex inhibitor is administered prior to the immunotherapy. In an embodiment, the Sec61 complex inhibitor is administered simultaneously with the immunotherapy.

The glioma cells may be glioblastoma cells. The subject may be a human.

In another aspect, provided herein is a method for inhibiting the Sec61 complex in a glioma cell, the method comprising contacting the cell with a Sec61 complex inhibitor. The inhibitor may reduce at least one of the expression of a Sec61 complex subunit and the activity of a Sec61 complex subunit. The Sec61 complex subunit may be Sec61G.

In an embodiment, the inhibitor comprises a small-molecular therapeutic agent. The inhibitor may be

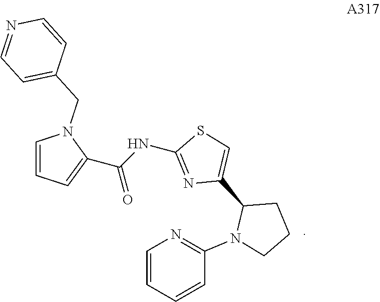 [SMILES: O=C(Nc1nc([C@H]2CCCN2c2ccccn2)cs1)c1cccn1Cc1ccncc1]

In an embodiment, the inhibitor comprises a genome editing system.

The method may further comprise contacting the cell with an immunotherapeutic agent for the glioma. In an embodiment, the cell is contacted with the Sec61 complex inhibitor prior to the immunotherapy. In an embodiment, the cell is contacted with the Sec61 complex inhibitor and the immunotherapy simultaneously.

### BRIEF DESCRIPTION OF THE DRAWINGS

The patent or patent application file contains at least one drawing in color. Copies of this patent or patent application publication with color drawings will be provided by the Office upon request and payment of the necessary fee.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1F. Genome-wide CRISPR-Cas9 knockout screen identifies SEC61G as gene involved in immunotherapeutic resistance that is highly expressed and heavily amplified in GBM. A CRISPR screen graphical. H4 glioma cells were transfected with the Brunello lentiviral library, expanded, and split between three groups: Day O (Baseline), Control, and Experimental. Healthy donor PBMCs were isolated and co-cultured at a 2:1 PBMC to H4 library cell ratio with or without Bi-specific T cells engaging (BiTE) antibody targeting IL13Rα2. After three repeated 72-hour treatments, viable cells were collected, DNA was isolated, barcoded via PCR, and PCR products were subjected to pooled paired-end next generation sequencing. B. Relative depleted guides (Blue-negative beta score) represent genes implicated in resistance while relative enriched guides (Red-positive beta score) represent genes implicated in sensitivity to T cell mediated killing. C. Analysis of TCGA-GBM database (bulk RNAseq). D. Genomic alteration of SEC61 G in three independent datasets: TCGA-GBM, MAYO-POX, and CPTAC. E. Single-cell analysis of SEC61 G expression in 110 GBM patients. F. Corresponding violin plot of single-cell gene expression by cell type.

FIGS. 2A-2E. Knockout of SEC61G sensitizes glioma cells to T-cell mediated killing. A. Confirmation of SEC61G KO in U87MG cells line. B. Flow cytometric analysis using anti-IL 13Rα2-PE conjugated Ab showed no difference in IL13Rα2 expression in U87 SEC61 G KO (U87-SEC61 G.KO) compared to the non-targeting control (U87-NTC) C. Cr51 release assay revealed a several-fold increase in cytotoxicity in response to targeted T-cell therapy in U87-SEC61G.KO compared to U87-NTC. D. Treatment of N10 and E. U87 glioma cells with SEC61 inhibitor A317 enhances responses to cytotoxic T cells in-co-culture assay.

FIGS. 3A-3E. SEC61 G expression influences the activity of MAPK pathway members. A. Heatmap of significantly differentially expressed genes between U87-SEC61G.KO and U87-NTC (control) from proteomic analysis. B. String pathway analysis of LRPPRC showing interconnection with MAPK pathway. C. PTPN11 directly connected to receptor tyrosine kinases. D. YWHAB and IQGAP1 proteins identified by mass spectrometry in SEC61 GKO U87MG glioma cells are connected to MAPK pathway. E. lmmunoblot analysis of MAPK activation following removal SEC61G in U87-SEC61G.KO in comparison to control U87-NTC cells.

FIGS. 4A-4D. GEM model recapitulates human GBM histological features. A Histological analysis of GBM from GEM models shows histological features similar to human disease, Positive stain for a target, IL13Rα2, sparsity of CD3+ T cells and the presence of CD11 b+ cells in tumor environment B. Response to BiTE treatment Log-rank test *p<0.05. C. ScRNAseq of CD45+-enriched cells from tumors of GEM model. D. SEC61G expression in glioma and myeloid clusters.

FIGS. 5A-5M. Modulation of SEC61G expression in TAMs using LPN encapsulated siRNAs. A-C. Umap of monocyte and TAMs clusters from GBmap (110 patients) comparing high vs. low SEC61 G expressing cells reveals upregulation of antigen-presentation pathways when SEC61 G expression is low, and vice versa. D. Analysis of SEC61 G expression in Gr1+ cells isolated from spleen and tumor of CT-2A murine glioma bearing mice (n=3). E. Murine TAMs were treated with lipid nanoparticles (LNP) LNP alone, LPNs encapsulated scrambled siRNA (siScramble), and SEC61 G siRNAs (siSec61 G). After 24 hr, total RNA was isolated and converted to cDNA. The RT-qPCR showed a nearly 90% knockdown of SEC61G in TAMs compared to all control conditions. Flow cytometric analysis using F-G. anti-CD206-FITC conjugated Ab and H-I. anti-lFNg-BV711-conjugated Ab showed a reduction in CD206 expression and increase in IFNg in TAMs treated with siSec61g compared to siScramble. Treatment of TAMs with SEC61-IN-1 inhibitor for 48 h at 25-100 ng/ml increases surface expression of J. MHC-I, but K. not MHCII class molecules, and L. decreases the expression of CD206 and M. PD-L1.

FIGS. 6A-6C. SEC6I-IN-1 penetrates in the brain and shows cytotoxicity to glioma cells at physiological relevant dose. A. LC-MS analysis of drug penetration of SEC61 complex inhibitor A317 (referred to as SEC61-IN-1) into mouse brain. B. MTT assay shows that SEC61 G-amplified GBM 12 and GBM38 response to SEC61 inhibitor at IC50 several folds lower than the concentration of inhibitor measured in the brain, whereas GBM6 without SEC61G amplification does not respond to the inhibitor treatment. C. EGFR amplification status shows wide range of SEC61G expression between PDX glioma lines.

### DETAILED DESCRIPTION

The present disclosure provides methods for targeting the Sec61 complex for sensitizing gliomas to immunotherapy.

### Methods

In a first aspect, provided herein is a method for sensitizing glioma cells to immunotherapy in a subject in need thereof, the method comprising administering to the subject an inhibitor of the Sec61 complex.

A glioma is a type of tumor that starts in the glial cells of the brain or the spine. About 33 percent of all brain tumors are gliomas, which originate in the glial cells that surround and support neurons in the brain, including astrocytes, oligodendrocytes and ependymal cells. In most cases, gliomas are cancerous. The glioma for treatment of the present invention may be an astrocytoma, a brain stem glioma, an ependymoma, a mixed glioma, an oligodendroglioma, an optic pathway glioma, etc. Astrocytomas are glial cell tumors developed from astrocytes and are the most common primary intra-axial brain tumor. High-grade astrocytomas, called glioblastoma multiforme (GBM), are the most malignant of all brain tumors. In exemplary embodiments, the glioma is a GBM.

As used herein, “a subject in need thereof” refers to a subject being treated with an immunotherapy for a glioma. When administered alone, the immunotherapy may be minimally effective, or unable to overcome the immunosuppressive environment of the glioma. The subject may be human.

The term “sensitizing” or “to sensitize” refers to increasing the response of the glioma cell to immunotherapy compared to its response in the absence of the Sec61 complex inhibitor. The response may include inhibition of cell growth and/or cell death.

The Sec61 complex is an endoplasmic reticulum membrane protein complex that transports proteins into the endoplasmic reticulum in eukaryotes. It is a doughnut-shaped pore through the membrane with three subunits, Sec61A, Sec61B and Sec61G. The Sec61 inhibitor may comprise an agent that targets the Sec61 complex, or one or more subunits of the Sec61 complex. As shown in the Examples, the inventors performed a genome-wide screen and identified SEC61G as a top gene contributing to the resistance of glioma cells to targeted T-cell therapy. Therefore, in some embodiments, Sec61G expression and/or activity is targeted for sensitizing glioma cells to immunotherapy. The inhibitor may target Sec61G.

The Sec61 complex inhibitor may be a small-molecular therapeutic agent. As used herein, the terms “small-molecular therapeutic agent”, “small-molecular compound”, and “small-molecule drug” refer to a chemical compound or pharmaceutically acceptable salt thereof having a therapeutic effect and/or enhancing the therapeutic effect of an immunotherapy. Small-molecule drugs are typically comprised of 20 to 100 atoms and have a molecular mass of less than 1000 g/mol or 1 kilodalton [kDa]. Small-molecules drugs can typically be administered by a variety of routes (including orally) and can pass through cell membranes to reach intercellular targets. The Sec61 complex inhibitor may be a small-molecular therapeutic agent that targets Sec61G. The small-molecular therapeutic agent may be

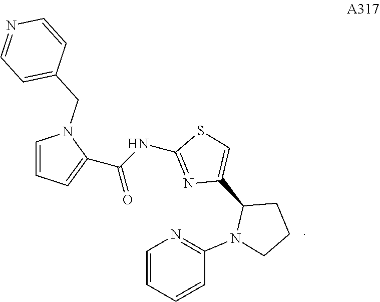 [SMILES: O=C(Nc1nc([C@H]2CCCN2c2ccccn2)cs1)c1cccn1Cc1ccncc1]

The small-molecular therapeutic agent may be any Sec61 targeting compound disclosed in US20220153732A1; US20230286973A1; U.S. Ser. No. 11/339,193B2; U.S. Ser. No. 11/578,055B2; US20220153732A1; WO2019046668A1; WO2023164250A1; Rehan et al. (2023) Signal peptide mimicry primes Sec61 for client-selective inhibition. Nature Chemical Biology, 19, 1054-1062; Wenzell et al. Global signal peptide profiling reveals principles of selective Sec61 inhibition. Nat Chem Biol. 2024 Mar. 22; Bade et al. 522 Quantitative proteomic profiling of KZR-540, a novel small molecule oral Sec61 inhibitor that selectively inhibits PD-1 expression. Journal for ImmunoTherapy of Cancer 2023, 11; clinicaltrials.gov/study/NCT05047536; kezarlifesciences.com/wp-content/uploads/Kezar-SITC-Poster-2023.pdf; kezarlifesciences.com/wp-content/uploads/Bade-ASMS-Poster-2022_5_YQ_DB_051222_JW_EL_051222_CK_mk2_YQ_with-qr-code_FINAL-003.pdf; kezarlifesciences.com/wp-content/uploads/KZR-8834-a-novel-small-molecule-inhibitor-of-sec6l.pdf; Foster et al. Pharmacological modulation of endothelial cell-associated adhesion molecule expression: implications for future treatment of dermatological diseases. J Dermatol. 1994 Nov;21(11):847-54; Chen et al. Solution-Phase Parallel Synthesis of a Pharmacophore Library of HUN-7293 Analogues: A General Chemical Mutagenesis Approach To Defining Structure-Function Properties of Naturally Occurring Cyclic (Depsi)peptides; J. Am. Chem. Soc. 2002, 124, 19, 5431-5440; Hommel et al. The 3D-structure of a natural inhibitor of cell adhesion molecule expression. Volume379, Issue 1, pages 69-73 (2016); Harant et al. The translocation inhibitor CAM741 interferes with vascular cell adhesion molecule 1 signal peptide insertion at the translocon. J Biol Chem. 2006 Oct. 13; 281(41):30492-502; Schreiner et al. Synthesis of ether analogues derived from HUN-7293 and evaluation as inhibitors of VCAM-1 expression. Bioorg Med Chem Lett. 2004 Oct. 4; 14(19):5003-6; Harant et al. Inhibition of vascular endothelial growth factor cotranslational translocation by the cyclopeptolide CAM741. Mol Pharmacol. 2007 June;71(6):1657-65; Garrison et al. A substrate-specific inhibitor of protein translocation into the endoplasmic reticulum. Nature 436, 285-289 (2005); Klein et al. Defining a Conformational Consensus Motif in Cotransin-Sensitive Signal Sequences: A Proteomic and Site-Directed Mutagenesis Study. PLoS ONE 10(3): e0120886; Westendorf et al. Inhibition of biosynthesis of human endothelin B receptor by the cyclodepsipeptide cotransin. J Biol Chem. 2011 Oct. 14; 286(41):35588-35600; Maifeld et al. Secretory Protein Profiling Reveals TNF-α Inactivation by Selective and Promiscuous Sec61 Modulators. Chemistry & Biology 18, 1082-1088, Sep. 23, 2011; Ruiz-Saenz et al. Targeting HER3 by interfering with its Sec61-mediated cotranslational insertion into the endoplasmic reticulum. Oncogene 34, 5288-5294 (2015); Junne et al. Decatransin, a new natural product inhibiting protein translocation at the Sec61/SecYEG translocon; J Cell Sci (2015) 128 (6): 1217-1229; Paatero et al. Apratoxin Kills Cells by Direct Blockade of the Sec61 Protein Translocation Channel. Cell Chem Biol. 2016 May 19;23(5):561-566; Kazemi et al. Targeting of HER/ErbB family proteins using broad spectrum Sec61 inhibitors coibamide A and apratoxin A. Biochemical Pharmacology. 2021 January;183:114317; Tranter et al. Coibamide A Targets Sec61 to Prevent Biogenesis of Secretory and Membrane Proteins; ACS Chem. Biol. 2020, 15, 8, 2125-2136; Kazemi et al. Targeting of HER/ErbB family proteins using broad spectrum Sec61 inhibitors coibamide A and apratoxin A. Biochemical pharmacology 183 (2021): 114317; Hall et al. Pleiotropic molecular effects of the Mycobacterium ulcerans virulence factor mycolactone underlying the cell death and immunosuppression seen in Buruli ulcer. Biochem Soc Trans. 2014 February;42(1):177-83; Morel et al. 2018. Proteomics reveals scope of mycolactone-mediated Sec61 blockade and distinctive stress signature. Molecular & Cellular Proteomics, 17(9), pp.l1750-1765; McKenna et al. Mechanistic insights into the inhibition of Sec61-dependent co- and post-translational translocation by mycolactone. J Cell Sci. 2016 Apr. 1; 129(7):1404-15; Demangel et al., 2018. Sec61 blockade by mycolactone: A central mechanism in Buruli ulcer disease. Biology of the Cell, 110(11), pp. 237-248; Mve-Obiang et al. A newly discovered mycobacterial pathogen isolated from laboratory colonies of Xenopus species with lethal infections produces a novel form of mycolactone, the Mycobacterium ulcerans macrolide toxin. Infect Immun. 2005 June;73(6):3307-12;

Zong et al. Ipomoeassin F Binds Sec61a to Inhibit Protein Translocation. J Am Chem Soc. 2019 May 29;141(21):8450-8461; O'Keefe et al. Ipomoeassin-F inhibits the in vitro biogenesis of the SARS-CoV-2 spike protein and its host cell membrane receptor. bioRxiv [Preprint]. 2021 Jan 5:2020.11.24.390039; Roboti et al. Ipomoeassin-F disrupts multiple aspects of secretory protein biogenesis. Sci Rep 11, 11562 (2021); Vermeire, 2002. CADA inhibits human immunodeficiency virus and human herpesvirus 7 replication by down-modulation of the cellular CD4 receptor. Virology, 302(2), pp. 342-353; Pauwels et al, 2021. A proteomic study on the membrane protein fraction of T cells confirms high substrate selectivity for the ER translocation inhibitor cyclotriazadisulfonamide. Molecular & Cellular Proteomics, 20; Claeys et al. Small Molecule Cyclotriazadisulfonamide Abrogates the Upregulation of the Human Receptors CD4 and 4-1BB and Suppresses In Vitro Activation and Proliferation of T Lymphocytes. Front Immunol. 2021 Apr. 21; 12:650731; Van Puyenbroeck et al. 2017. A proteomic survey indicates sortilin as a secondary substrate of the ER translocation inhibitor cyclotriazadisulfonamide (CADA). Molecular & Cellular Proteomics, 16(2), pp.157-167; Cross et al. Eeyarestatin I inhibits Sec61-mediated protein translocation at the endoplasmic reticulum. J Cell Sci (2009) 122 (23): 4393-4400; Lowe, Eric, et al. “Preclinical evaluation of KZR-261, a novel small molecule inhibitor of Sec6L.” (2020): 3582-3582; each of which are incorporated herein by reference.

The small-molecular therapeutic agent may contain a 5-membered heterocyclic core. In some embodiments, the heterocyclic core contains a thiazole. The thiazole may be substituted with an amide linker. For example, the small-molecular therapeutic agent may contain a pyridine-alkylene (optional)-pyrrole-amide-thiazole moiety. In some embodiments, the heterocyclic core contains an imidazolidine derivative. In some embodiments, the small-molecular therapeutic agent

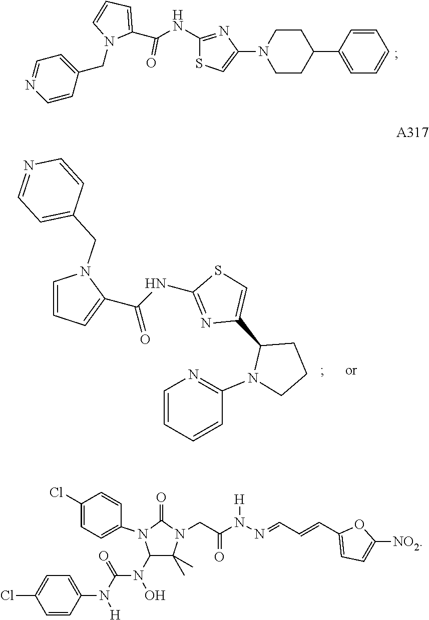 [SMILES: O=C(Nc1nc(N2CCC(c3ccccc3)CC2)cs1)c1cccn1Cc1ccncc1 ; O=C(Nc1nc([C@H]2CCCN2c2ccccn2)cs1)c1cccn1Cc1ccncc1 ; CC1(C)C(N(O)C(=O)Nc2ccc(Cl)cc2)N(c2ccc(Cl)cc2)C(=O)N1CC(=O)N/N=C/C=C/c1ccc([N+](=O)[O-])o1]

The small-molecular therapeutic agent may contain a macrocyclic structure. “Macrocycle” refers to rings of at least 10, at least 11, at least 12, or more atoms. In some embodiments, the small-molecular therapeutic agent contains a macrocyclic peptide core, such as a macrocyclic depsipeptide core. In some embodiments, the small-molecular therapeutic agent contains a macrolide core. In some embodiments, the small-molecular therapeutic agent contains a cyclotriazasulfonamide core. In some embodiments, the small-molecular therapeutic agent is a natural product or a natural product derivative, such as:

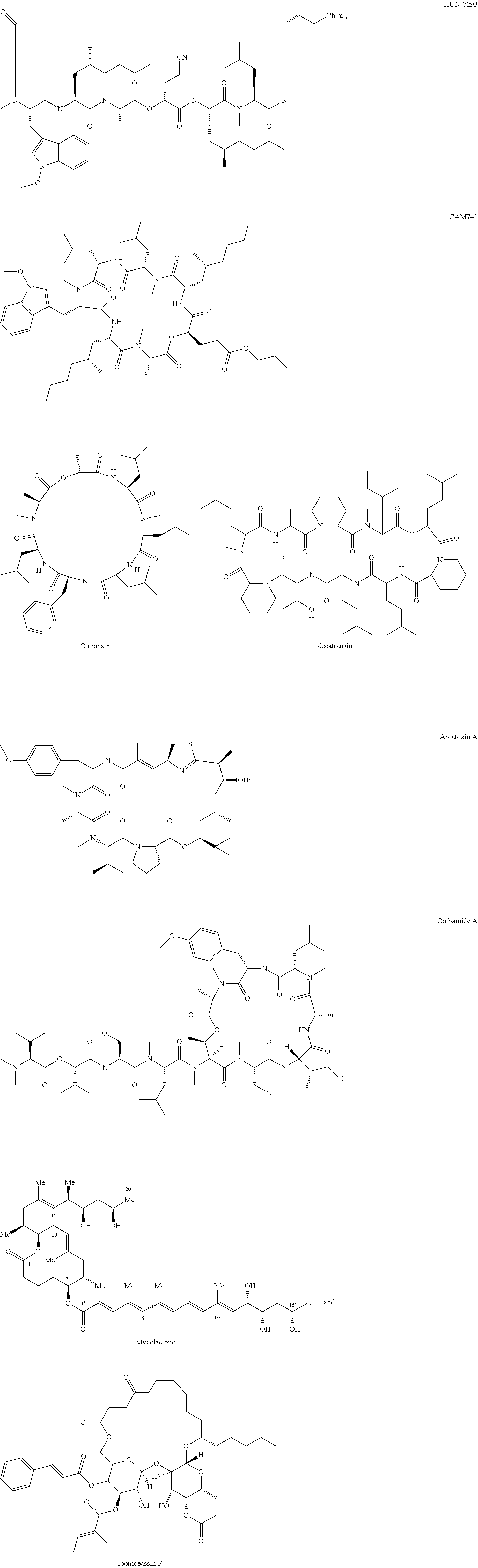

The small-molecular therapeutic agent may be:

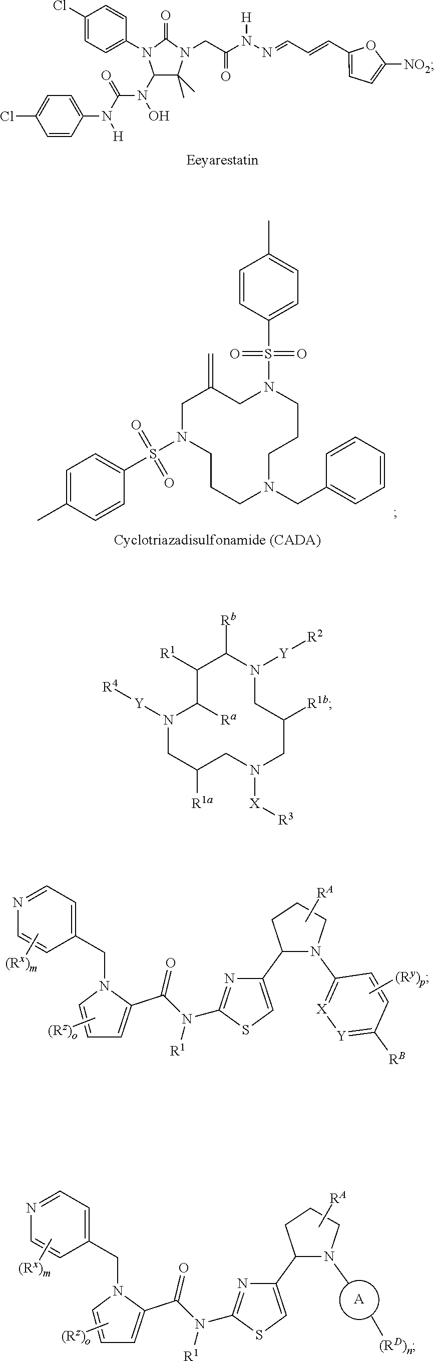

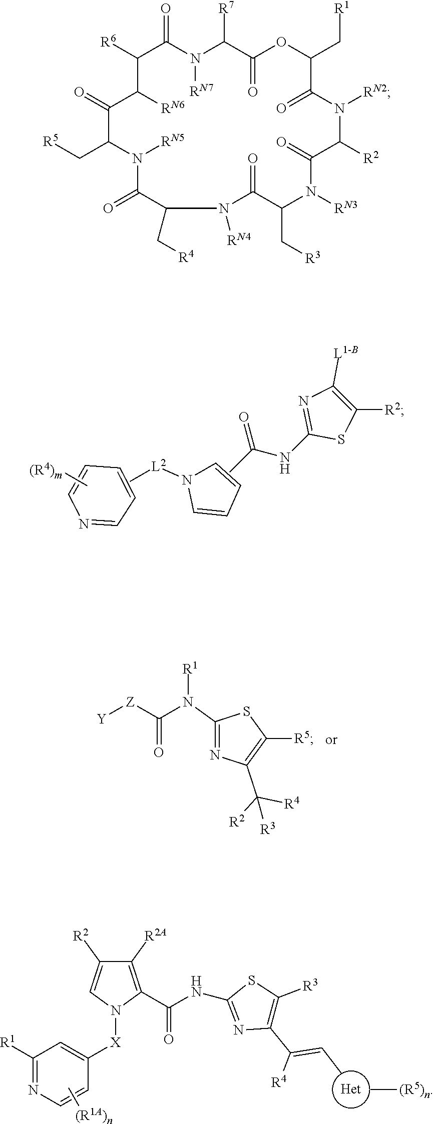

The small-molecular therapeutic agent may be:

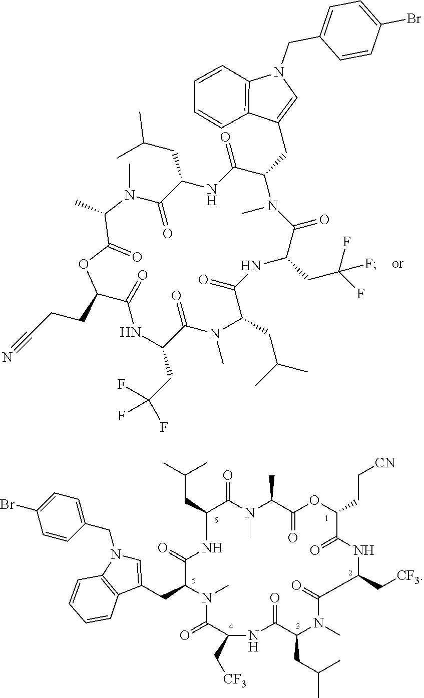

The Sec61 complex inhibitor may comprise a genome editing system. The genome editing system may delete, modify, or replace portions of DNA encoding a Sec61 complex subunit, e.g. Sec61G, or a protein that regulates the subunit. The genome editing system may comprise any genetic engineering technique known in the art. The system may comprise an engineered nuclease, including, but not limited to a Cas nuclease, a zinc finger nuclease (ZFN), or a transcription activator-like effector-based nuclease (TALEN).

Clustered Regularly Interspaced Short Palindromic Repeats (CRISPR)/Cas gene editing systems have been developed to enable targeted modifications to a specific gene of interest in eukaryotic cells. CRISPR/Cas gene editing systems are based on the RNA-guided Cas9 nuclease from the type II prokaryotic clustered regularly interspaced short palindromic repeats (CRISPR) adaptive immune system. (See, e.g., Jinek et al., Science, 337: 816 (2012); Gasiunas et al., Proc. Natl. Acad. Sci. U.S.A., 109, E2579 (2012); Garneau et al., Nature, 468: 67 (2010); Deveau et al., Annu. Rev. Microbiol., 64: 475 (2010); Horvath and Barrangou, Science, 327: 167 (2010); Makarova et al., Nat. Rev. Microbiol., 9, 467 (2011); Bhaya et al., Annu. Rev. Genet., 45, 273 (2011); Cong et al., Science, 339: 819-823 (2013); and U.S. Pat. Nos. 8,697,359; 8,795,965; and 9,322,037). The CRISPR/Cas9 system is extensively used in the art to edit the genome of zygotes to generate various genetically modified animal species, including mice, and rats. The use of CRISPR/Cas9 in postnatal or adult animals including canines and monkeys also is under investigation. (See Cong et al., Science 339, 819-823 (2013); Cox et al., supra, Doudna et al., Science, 346: 1258096 (2014); and Yin et al., supra).

Other CRISPR/Cas systems known in the art may be used in the methods, including, for example, CRISPR/Cas13, which induces RNA knockdown. (Zetsche et al., Cell, 163: 759-771 (2015)) and CRISPR/Cpf129 (Kim et al., Nature Communications, 8 (14406): 14406 (2017)), and base editors (Gehrke et al., Nature Biotechnology, 37: 224-226 (2019)), and prime editing (Anzalone et al., Nature, 576: 149-157 (2019)). CRISPR/Cas systems suitable for use in connection with the present disclosure are further described in, e.g., Marakova, K. S. and E. V. Koonin, Methods Mol. Biol., 1311: 47-75 (2015); Sander et al., Nat. Biotechnol., 32(4): 347-55 (2014); and Gootenberg et al., Science, 356(6336): 438-442 (2017)).

These CRISPR/Cas systems use guide RNAs (gRNAs) that direct Cas enzymes to cleave targeted DNA or RNA. The genome editing system employed in the methods described herein utilize Cas enzymes directed by gRNAs that target and cleave the nucleic acid encoding the Sec61 complex subunit.

The Sec61 complex inhibitor may comprise a gene silencing agent. The gene silencing agent may reduce expression of a Sec61 complex subunit or a molecule that positively regulates a subunit. The gene silencing agent may reduce expression of Sec61G or a molecule that positively regulates Sec61G. The gene silencing agent may comprise a nucleic acid sequence that is capable of inducing RNA interference (RNAi). The term “RNA interference” refers to a process in which RNA molecules inhibit gene expression or translation by neutralizing targeted mRNA molecules. To achieve an RNAi effect, for example, RNA having a double strand structure containing the same base sequence as that of the target mRNA may be used. Two types of small RNA molecules may induce RNAi: microRNA (miRNA) and small interfering RNA (siRNA). miRNA is a small non-coding RNA molecule (typically containing about 20-25 nucleotides) found in plants, animals and some viruses, which silences complementary target sequences by one or more of the following processes: (1) cleavage of the target mRNA strand into two pieces, (2) destabilization of the mRNA through shortening of its poly(A) tail, and (3) less efficient translation of the mRNA into proteins by ribosomes. (See Bartel D.P., Cell, 136 (2): 215-233 (2009); and Fabian et al., Annual Review of Biochemistry, 79: 351-79 (2010)). siRNA (also known as short interfering RNA or silencing RNA), is a class of double-stranded RNA molecules, typically 20-25 base pairs in length, which silence complementary target sequences by degrading mRNA after transcription, preventing translation. (See Dana et al., International Journal of Biomedical Science, 13(2):48-57 (2017); and Agrawal, et al., Microbiol. Mol. Biol. Rev., 67: 657-685 (2003)). siRNA can also act in RNAi-related pathways in an antiviral mechanism or play a role in the shaping of the chromatin structure of a genome. Any RNA molecule that is capable of silencing gene expression of a target gene may be used in connection with the present disclosure. In some embodiments, the RNA molecule is siRNA, miRNA, antisense oligoes. In other embodiments, the RNA molecule may a long non-coding RNA (lncRNA). Long non-coding RNAs are a large and diverse class of transcribed RNA molecules with a length of more than 200 nucleotides that do not encode proteins. lncRNAs are thought to encompass nearly 30,000 different transcripts in humans, and account for the major part of the non-coding transcriptome. While the mechanism of action of lncRNAs is under investigation, lncRNAs appear to be important regulators of gene expression, and lncRNAs are thought to have a wide range of functions in cellular and developmental processes. lncRNAs may carry out both gene inhibition and gene activation through a range of diverse mechanisms (see, e.g., Kung et al., Genetics, 193(3): 651-666 (2013); and Marchese et al., Genome Biol., 18: 206 (2017)).

As used herein, the term “administering”, refers to dispensing, delivering, or applying the substance, e.g. a Sec61 complex inhibitor or an immunotherapeutic agent, to a subject by any suitable route for delivery of the substance to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The methods may further comprise administering an immunotherapy or immunotherapeutic agent for the glioma cells to the subject. The Sec61 complex inhibitor may be administered prior to or simultaneously with the immunotherapy treatment. The term “immunotherapy” refers to treatment of disease by activating or suppressing the immune system. For example, immunotherapy as applied to a cancer relevant to the present application may include the use of agents that target checkpoint inhibition (e.g., immune checkpoint inhibitors). For instance, the immunotherapy may include agents (e.g., antibodies (neutralizing antibodies), small-molecule/chemical compound inhibitors, protein-binding peptides) that bind and neutralize the action of immune checkpoint proteins that regulate the immune response. The neutralization of immune checkpoint proteins can lead to increased activity of T cells, resulting in increased targeting and killing of cancer cells. Immunotherapy agents include any type of antibody or antibody-like peptides including but not limited to nanobodies, fragmented antibodies or fragmented antibody derivatives (e.g., Fab, Fab′, F(ab′)2, sdAb, scFv, di-scFv), peptide/MHC-complexes, cell adhesion receptor molecules, receptors for costimulatory molecules, and artificial engineered binding molecules (e.g., aptamers). In embodiments, the immune checkpoint inhibitors target one or more proteins involved in the immune checkpoint pathway. The one or more targeted immune checkpoint proteins may include but not be limited to PD-1, PD-L1, and CTLA-4. For example, a therapy may include the administration of an antibody or small molecular compound against PD-1, PD-L1, or CTLA-4 to a subject for the treatment of cancer.

The terms “treating” and “to treat” includes the reducing, repressing, delaying or preventing cancer (e.g. glioma) growth, reduction of tumor volume, and/or preventing, repressing, delaying or reducing metastasis of the tumor. Treating may also include reducing the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; (d) reducing or ameliorating at least one symptom of cancer; and (e) extending the survival of the subject. In some embodiments, the optimum effective amount can be readily determined by one skilled in the art using routine experimentation.

The Sec61 complex inhibitor and/or the immunotherapeutic agent may be prepared as a formulation or pharmaceutical composition. Inert ingredients and manner of formulation of the pharmaceutical compositions are conventional. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York). The pharmaceutical compositions may be designed or intended for oral, rectal, nasal, systemic, topical or transmucosal (including buccal, sublingual, ocular, vaginal and rectal) and parenteral (including subcutaneous, intramuscular, intravenous, intraarterial, intradermal, intraperitoneal, intrathecal, intraocular and epidural) administration. In embodiments, aqueous and non-aqueous liquid or cream formulations are delivered by a parenteral, oral or topical route. In embodiments, the compositions may be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal or intramuscular depot. The pharmaceutical compositions may be lyophilized. The pharmaceutical compositions may contain auxiliary substances such as wetting or emulsifying agents, pH buffering agents, gelling or viscosity enhancing additives, preservatives, flavoring agents, colors, and the like, depending upon the route of administration and the preparation desired. For intravenous administration, suitable carriers include physiological saline, bacteriostatic water, Cremophor EL® (BASF, Parsippany, N.J., USA) or phosphate buffered saline (PBS). In all cases, a composition for parenteral administration must be sterile and should be formulated for ease of injectability. The composition should be stable under the conditions of manufacture and storage, and must be shielded from contamination by microorganisms such as bacteria and fungi. The composition may further comprise a pharmaceutically acceptable carrier. The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active chemical compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Capsules are prepared by mixing the chemical compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders. Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The chemical compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects. A lubricant can be used in a tablet formulation to prevent the tablet and punches from sticking in the die. The lubricant can be chosen from such slippery solids as talc, magnesium and calcium stearate, stearic acid, and hydrogenated vegetable oils. Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the chemical compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The preferred route may vary with, for example, the subject's pathological condition or age or the subject's response to therapy or that is appropriate to the circumstances. The formulations can also be administered by two or more routes, where the delivery methods are essentially simultaneous, or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

The terms “effective amount” or “therapeutically effective amount” refer to an amount sufficient to effect beneficial or desirable biological and/or clinical results. The amount of the pharmaceutical composition that is therapeutically effective may vary depending on the particular pathogen or the condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In a second aspect, provided herein is a method for inhibiting the Sec61 complex in a glioma cell, the method comprising contacting the cell with a Sec61 complex inhibitor. “Contacting” as used herein, e.g., as in “contacting a cell” or “contacting a sample” refers to contacting a sample directly or indirectly in vitro or ex vivo. Further, contacting a cell includes adding an agent to a cell culture. The method may further comprise contacting the cell with an immunotherapeutic agent for the glioma.

### Compositions

In a third aspect, provided herein is a composition comprising a Sec61 complex inhibitor and an immunotherapeutic agent. The compositions may be prepared as described above.

### Kits

In a fourth aspect, provided herein is a kit comprising a first pharmaceutical composition comprising a Sec61 complex inhibitor and a second pharmaceutical composition comprising an immunotherapeutic agent. The compositions may be prepared as described above.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Example

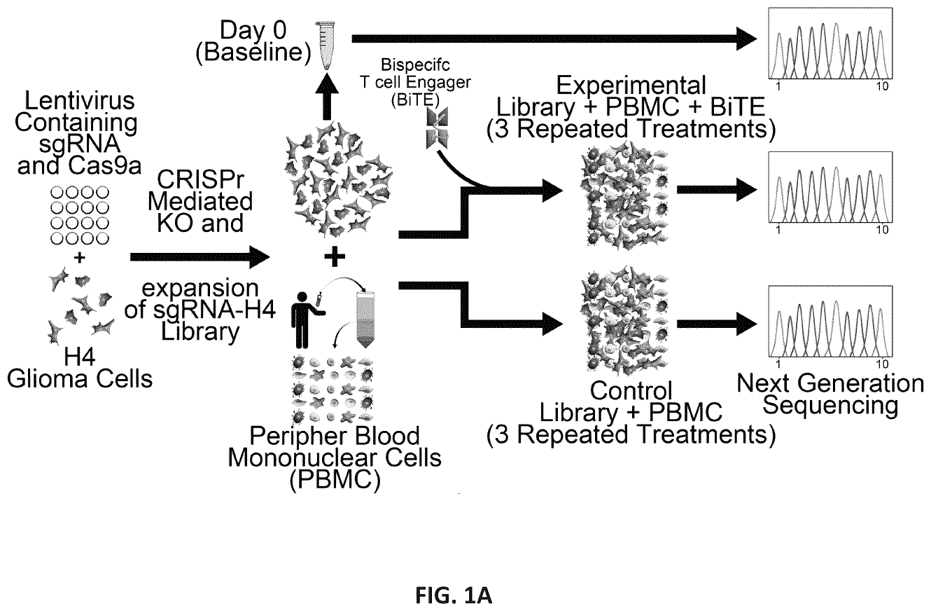

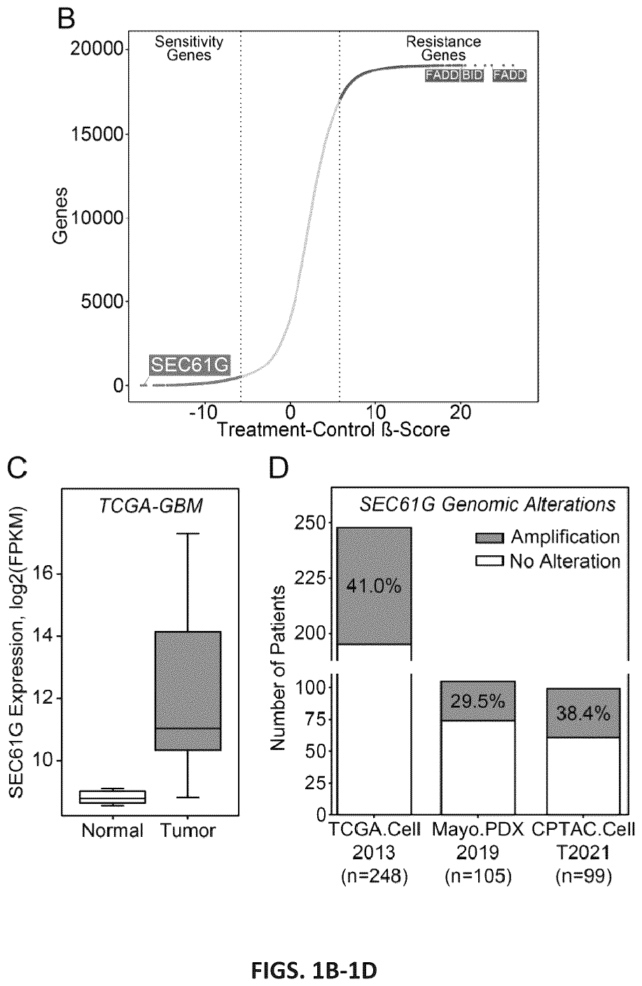

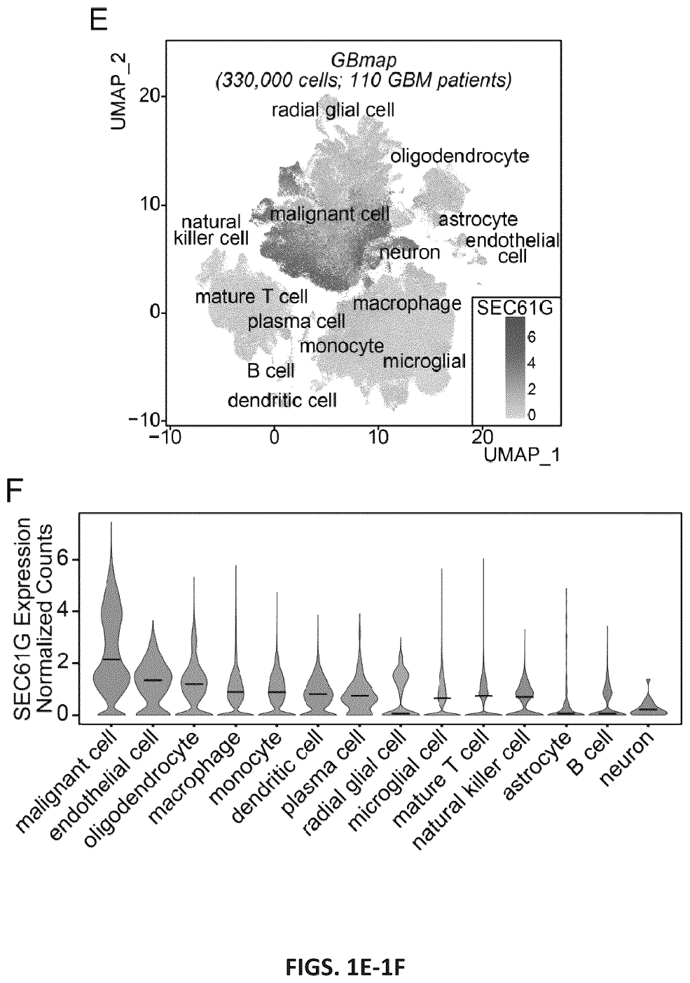

To identify genes contributing to immunotherapeutic resistance in glioblastoma (GBM), we performed an unbiased, genome-wide CRISPR knockout screen in a glioma cell line, H4, using targeted T cell therapy (bi-specific T cell engager) as the selection pressure (FIG. 1A).

We performed the bioinformatic analysis to compare genetic fluctuations in the experimental group compared to the control. The SEC61G was identified as a top gene to contribute to the resistance of glioma cells to targeted T-cell therapy (FIG. 1B). SEC61G, along with SEC61A1/2, and SEC61B, function in a hetero-trimeric translocon complex known as the SEC61 complex. Residing within the endoplasmic reticulum, the SEC61 complex plays an essential role in channeling precursors and modifying polypeptides.

Subsequent transcriptomic analysis of patient samples from the TCGA-GBM dataset showed overexpression of SEC61G in GBM compared to normal brain tissue (FIG. 1C). Further analysis of TCGA-GBM, Mayo GBM PDX, and the National Cancer Institute's Clinical Proteomic Tumor Analysis Consortium (CPTAC) datasets demonstrated genomic amplification of SEC61G in 41.0, 29.5, and 38.4% of GBM patients, respectively (FIG. 1D). Analysis of a harmonized-single cell RNA sequencing dataset of 110 GBM patient samples (doi: https://doi.org/10.1101/2022.08.27.505439) revealed SEC61G expression is predominantly restricted to glioma cells but also found in other cells of the tumor microenvironment (FIGS. 1E-1F).

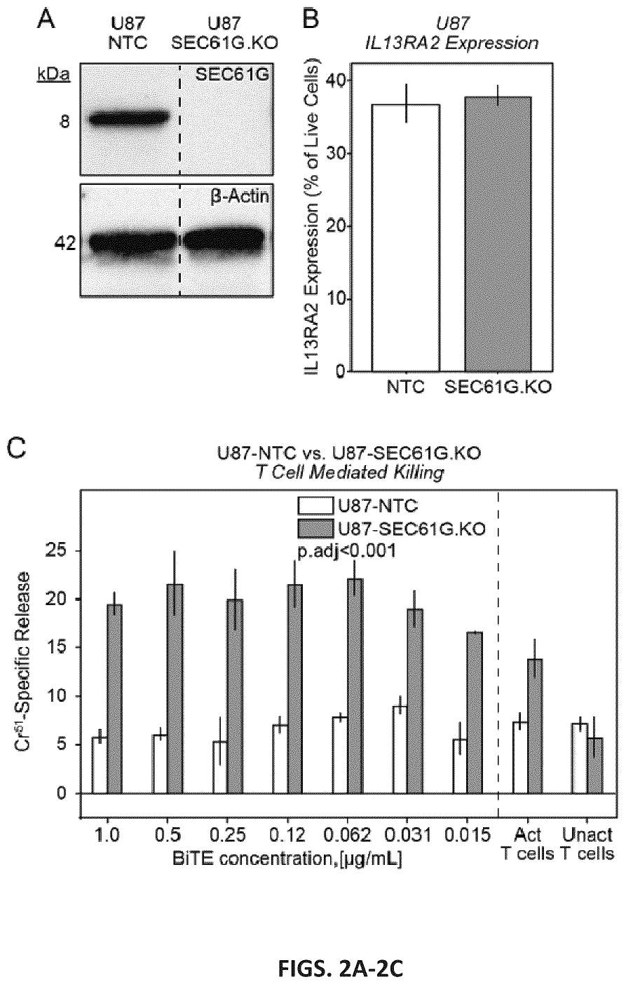

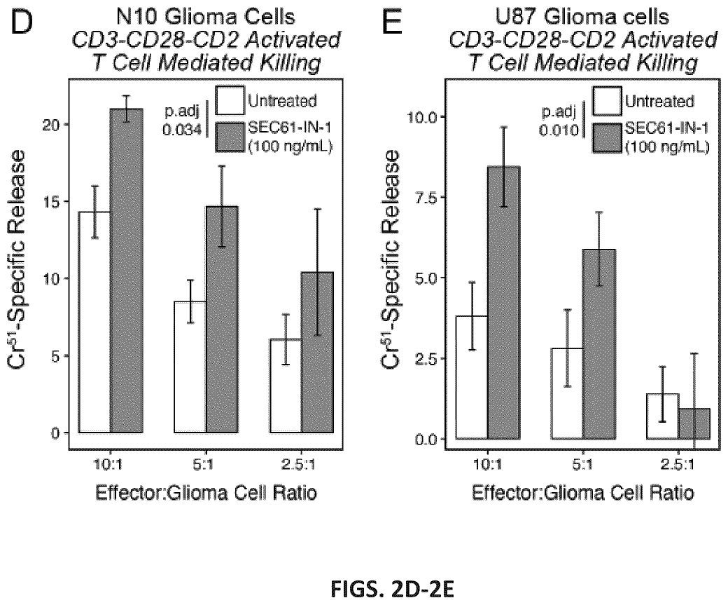

To confirm the screen results, we used the CRISPr-cas9 system to knockout SEC61G in another Glioma cell line relatively resistant to targeted immunotherapy, U87-MG (FIG. 2A). The expression of the BiTE target, IL13Rα2, was not altered in SEC61G.KO cells (FIG. 2B). CRISPr-cas9 mediated knockout CA Cr51 release cytotoxicity assay revealed a several-fold increase in response to targeted T-cell therapy in U87-MG SEC61G knockout cells (U87-SEC61G.KO) compared to their respective U87-MG non-targeting control cells (U87-NTC) (FIG. 2C). We further validated our finding is N10 and U87 glioma cells, where N10 (FIG. 2D) and U87 glioma cells (FIG. 2E) became more responsive to CD3/CD28/CD2-bead-activated T cells following pretreatment with SEC61-IN-1 (e.g., A317, developed by Kezar Life Sciences).

To uncover mechanistic pathways by which SEC61G influences immunotherapeutic response, we performed a bottom-up proteomic analysis of U87-SEC61G.KO. Differential protein

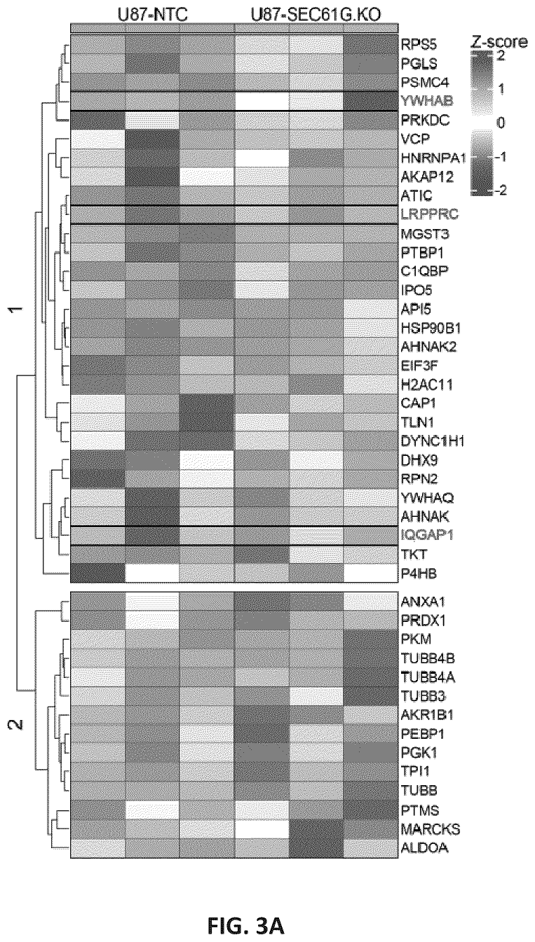

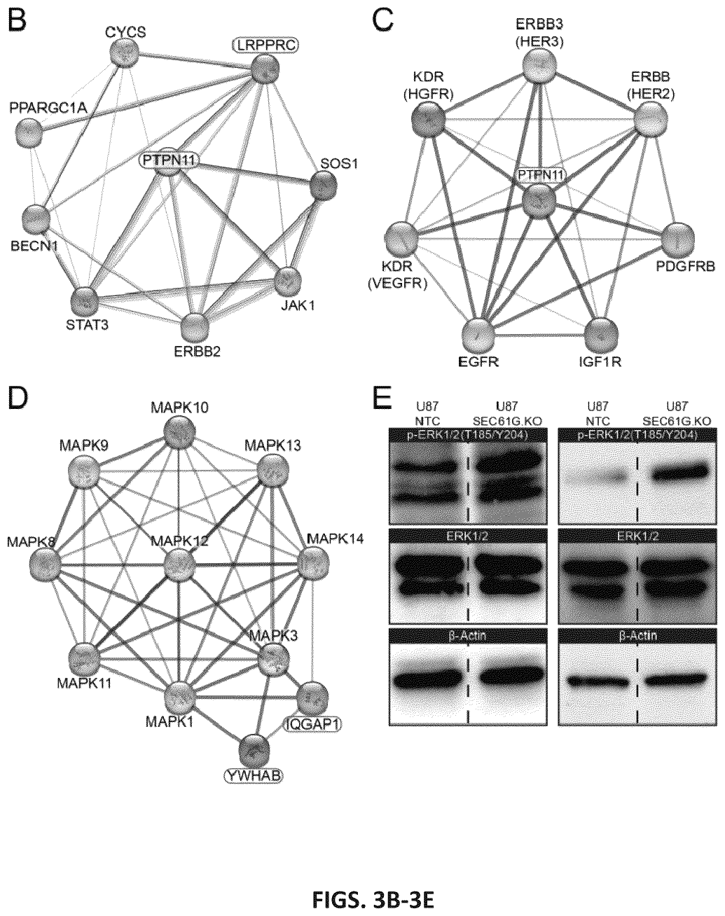

analysis revealed 43 differentially expressed proteins, 29 of which were upregulated, in U87-SEC61G.KO compared to U87-NTC (FIG. 3A). Several significantly upregulated proteins were connected to MAPK pathway, such as LRPPRC (FIG. 3B), PTPN11 (FIGS. 3B-3C), YWHAB (FIG. 3D), and IQGAP1 (FIG. 3D). Through immunoblotting, we subsequently confirm a several-fold higher MAPK1/3 activation in U87-SEC61G.KO compared to U87-NTC (FIG. 3E).

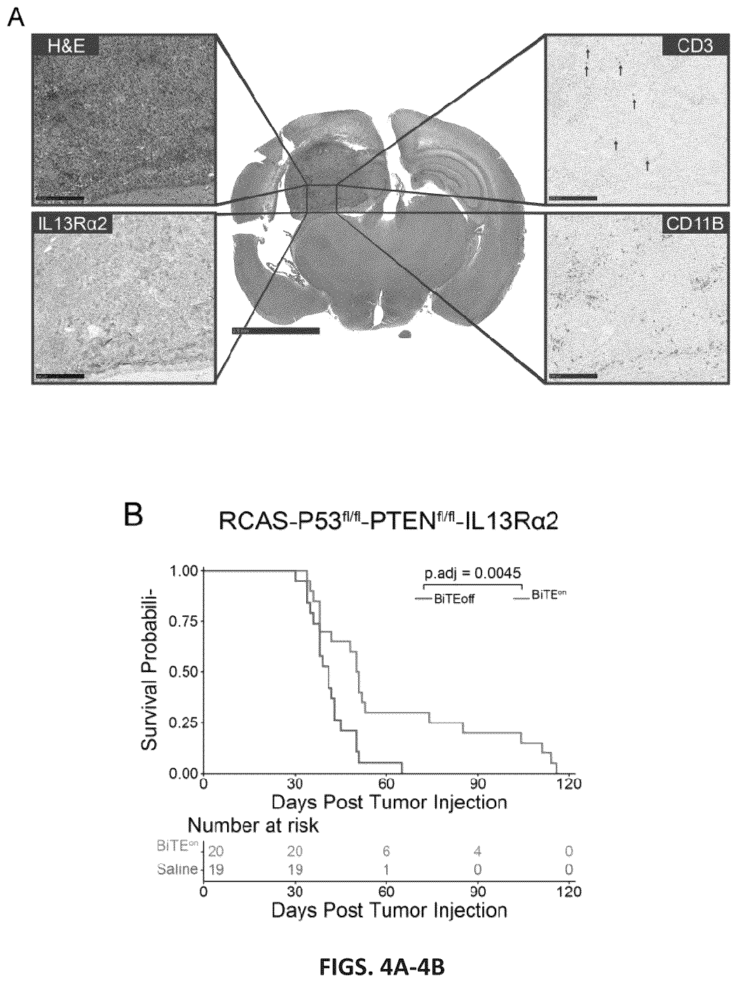

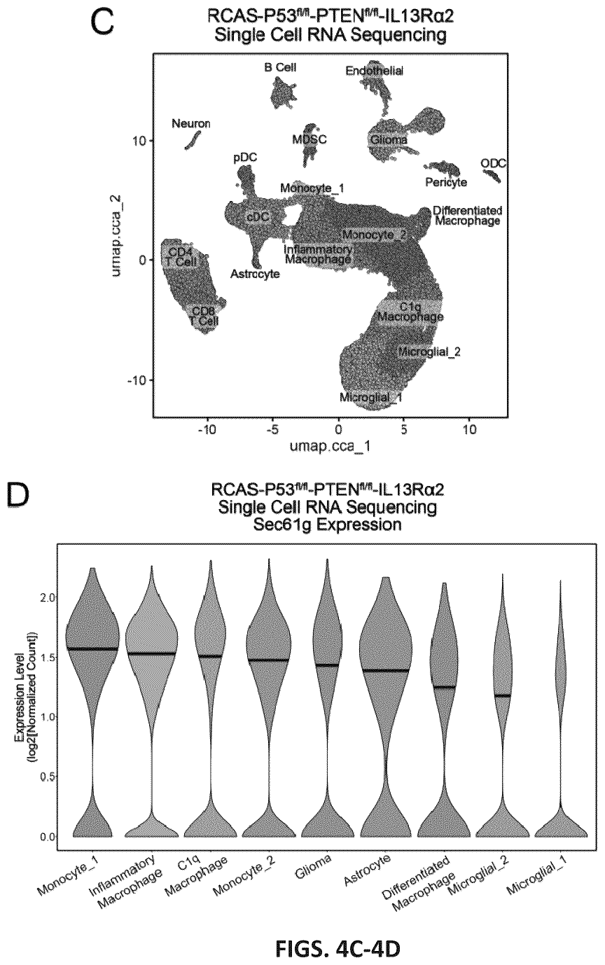

Histological staining of GEM of GBM expressing IL13Rα2 in adult mice derived from the established PDGFB/p53−/− PTEN−/− RCAS/Tv-a model^{1,2}. GEM displayed hallmarks of GBM with cellular invasion, pseudo-palisading necrosis and areas of microvascular proliferation within a tumor. Tumors displayed features of a “cold” immunological landscape, high infiltration of CD11b+ myeloid cells, and poor infiltration of CD3+ T cells (FIG. 4A). This model was moderately responsive to IL13RA2 targeted BiTE therapy (FIG. 4B). Single cell RNAseq of CD45 enriched cells (8:2 ratio of CD45pos:CD45neg) revealed a highly heterogeneous PDGFB/p53−/− PTEN−/− RCAS/Tv-a tumor microenvironment dominated by both resident and peripheral myeloid cells (FIG. 4C). Both glioma cells and myeloid subsets highly expressed SEC61G (FIG. 4D). Thus, this model provides a valuable tool for studying the SEC61G mediated resistance to immunotherapy in GBM.

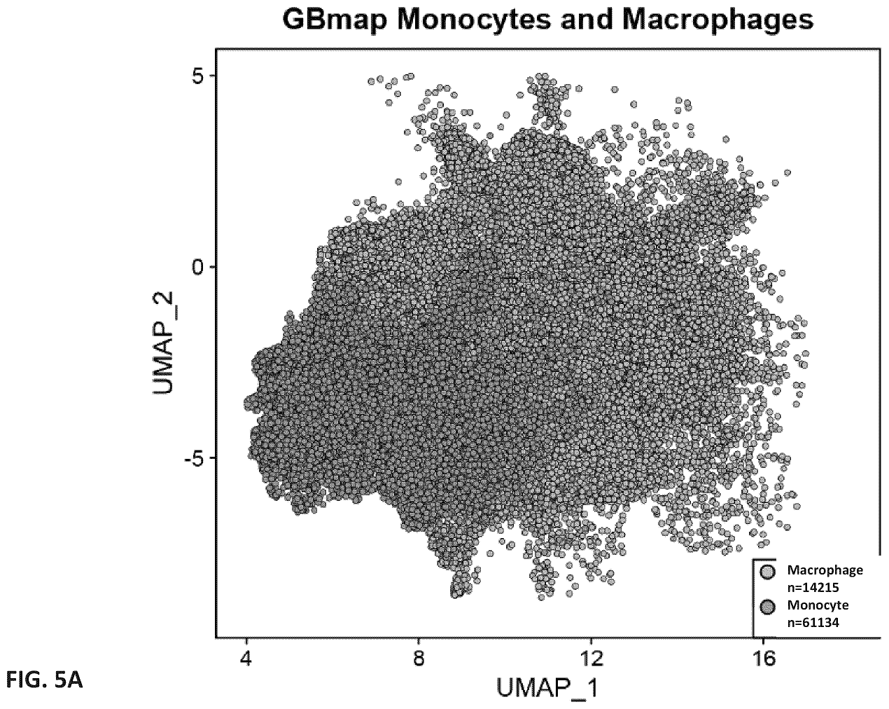

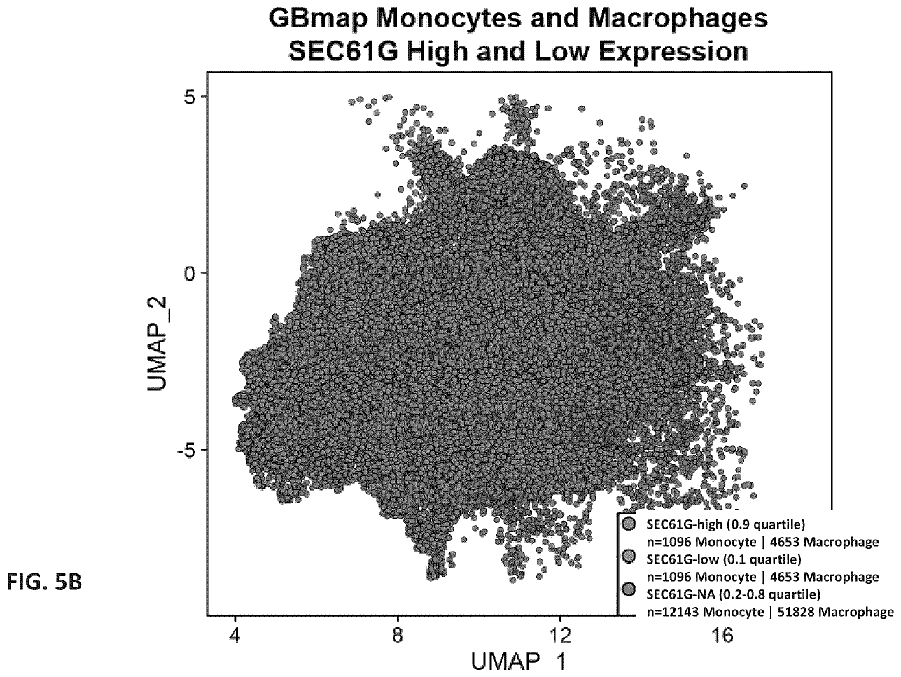

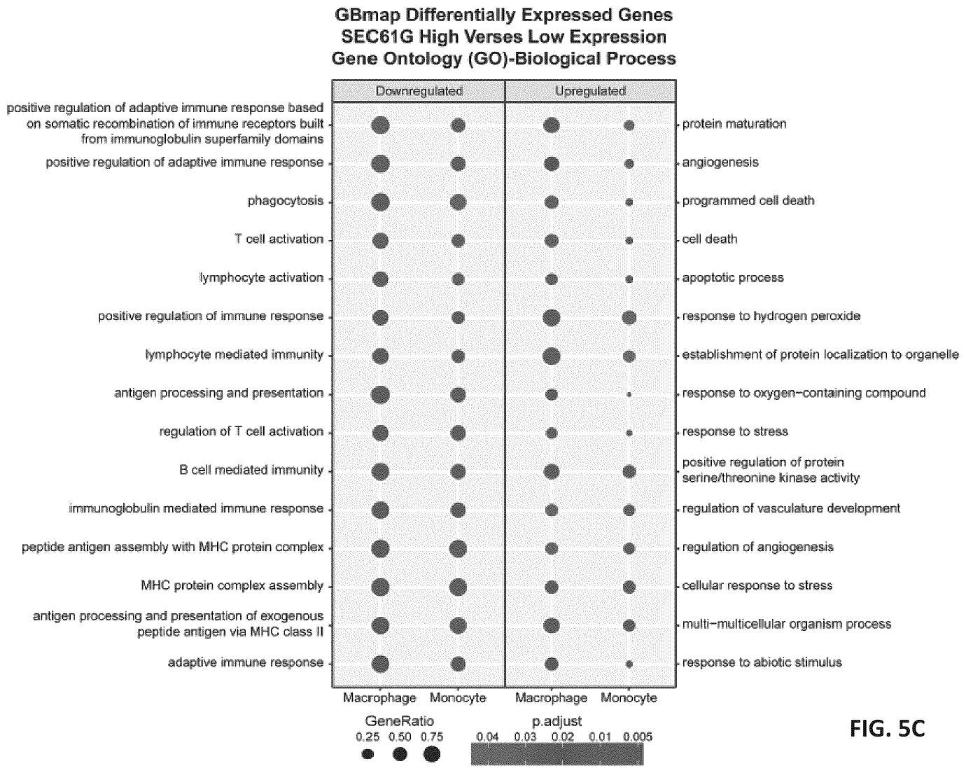

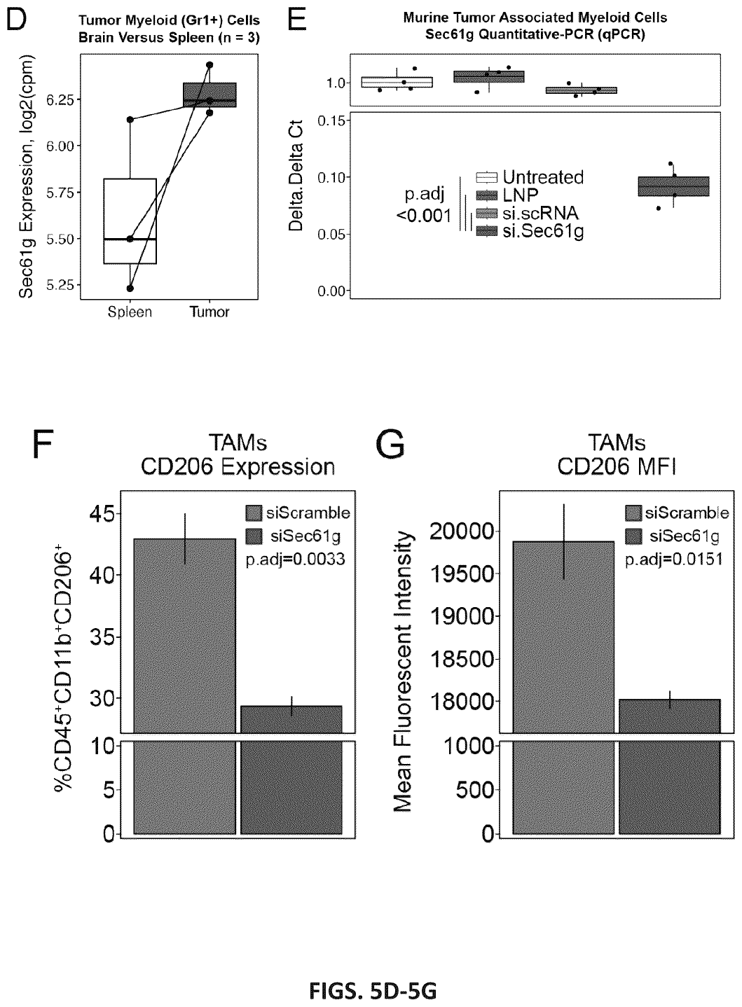

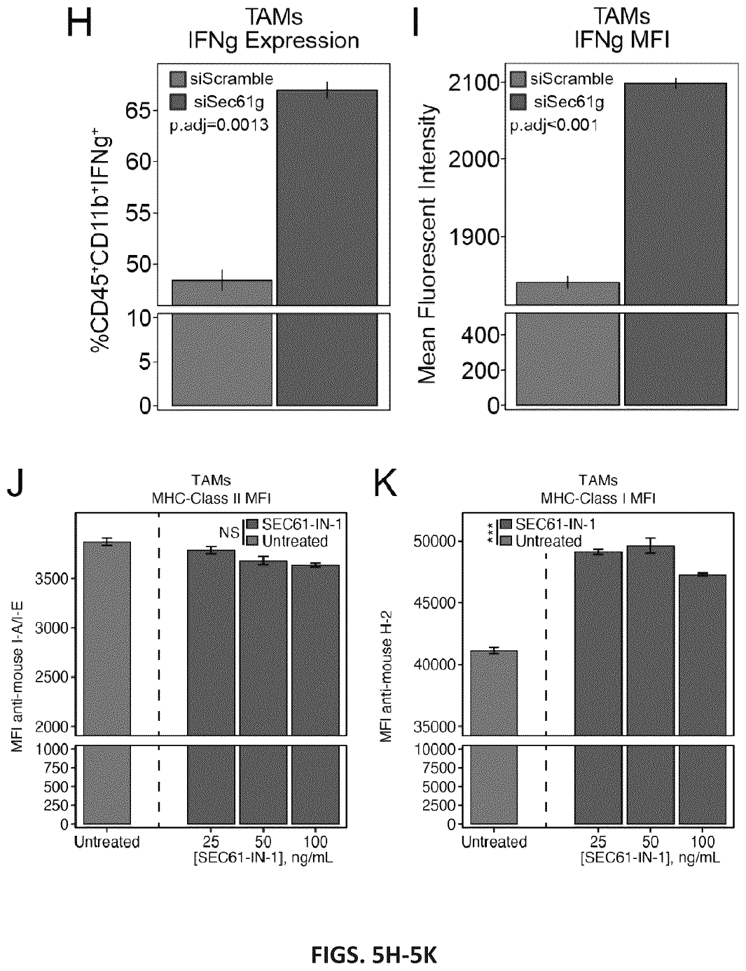

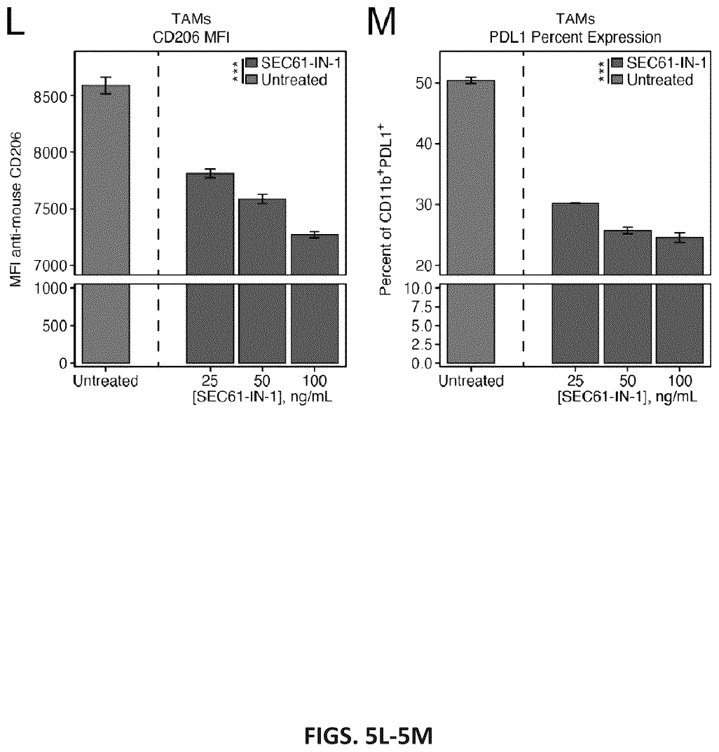

Tumor-associated myeloid cells (TAMs) comprise up to 50% of GBM tumor mass, and this myeloid-dominated, highly immunosuppressive TME plays a profound role in resistance to immunotherapies³. Single-cell RNAseq analysis revealed SEC61G expression in the TAMs compartment of both human and mouse GBM tissue4. While SEC61G expression prevails in glioma cells over TAMs in human GBM (FIGS. 1E-1F), subsetting TAM clusters (FIG. 5A) from scRNA seq data set of 110 GBM patients for differential gene expression between high vs. low SEC61G expressing cells (FIG. 5B) revealed significant downregulation of proteins involved in antigen-presentation in SEC61G high-expressing cells (FIG. 5C). To better understand this relationship, we elected to test knockdown and inhibition of SEC61G in murine myeloid cells. We first confirmed SEC61G using RNA sequencing of Gr1+ cells isolated from spleen and tumor of CT-2A murine glioma bearing mice (n=3) (FIG. 5D). Murine TAMs were then treated with LPN alone, LPNs encapsulated scrambled siRNA (siScramble), and SEC61G siRNAs (siSec61g) and qRT-PCR showed a nearly 90% knockdown of SEC61G compared to all control conditions (FIG. 5E). Flow cytometric analysis revealed reduced expression of the immunosuppressive marker CD206 (FIG. 5F), and increased expression of IFNg (FIG. 5G) in TAMs treated with siSec61g compared to siScramble. We also observed similar results using SEC61-IN-1 inhibitor, with increased surface expression of MHC-I (FIG. 5J), but not MHC-II class molecules (FIG. 5K), and decreased expression of CD206 (FIG. 5L) and PD-L1 (FIG. 5M).

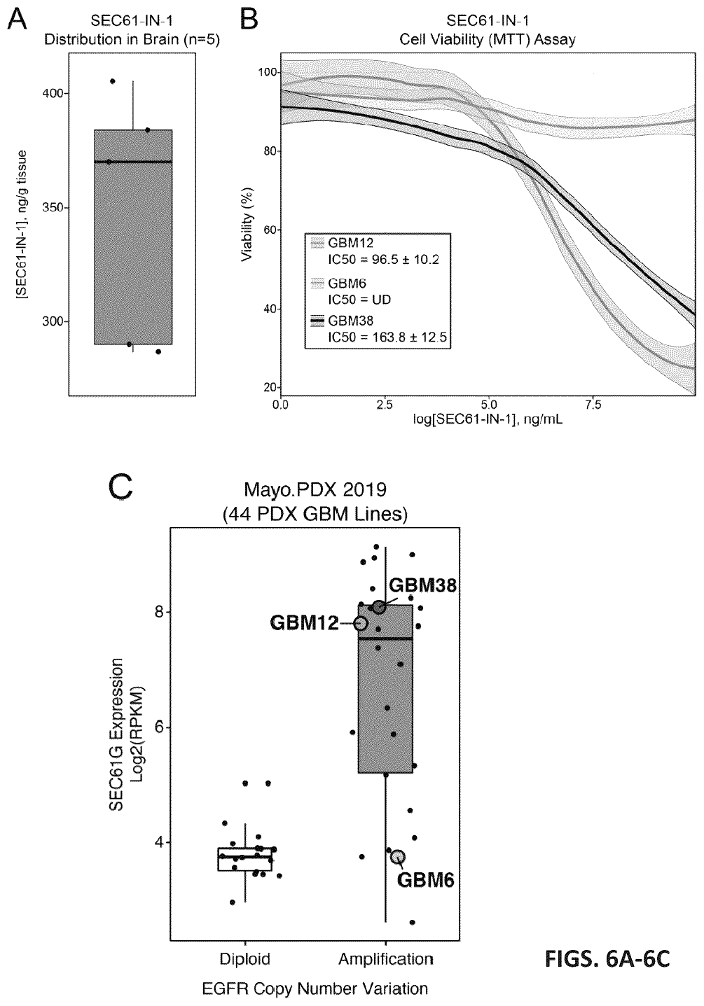

To test SEC61-IN-1 (e.g. A317, Kezar Life Sciences) penetration, we performed LC-MS analysis of drug penetration into mouse brain. We detected 400 ng/g tissue (5-7% of plasma concentration) 45 min post a single injection of inhibitor at 25 mg/kg (FIG. 6A). We then went on to test the cytotoxic activity of SEC61-IN-1 in dual SEC61G-EGFR-amplified GBM12 and GBM38, and EGFR-amplified GBM6 (not SEC61G-amplified) PDX cell lines (FIG. 6B). We found response to SEC61 inhibitor at IC50 several folds lower than the concentration of inhibitor measured in the brain in SEC61G-amplified GBM12 and GBM38, but not GBM6 (FIG. 6C).

### REFERENCES

1 Hambardzumyan, D., Amankulor, N. M., Helmy, K. Y., Becher, O. J. & Holland, E. C. Modeling Adult Gliomas Using RCAS/t-va Technology. Transl Oncol 2, 89-95 (2009). https://doi.org/10.1593/tlo.09100
2 Misuraca, K. L., Cordero, F. J. & Becher, O. J. Pre-Clinical Models of Diffuse Intrinsic Pontine Glioma. Front Oncol 5, 172 (2015). https://doi.org/10.3389/fonc.2015.00172
3 Pombo Antunes, A. R. et al. Understanding the glioblastoma immune microenvironment as basis for the development of new immunotherapeutic strategies. Elife 9 (2020). https://doi.org/10.7554/eLife.52176
4 Cristian, R.-M. et al. Harmonized single-cell landscape, intercellular crosstalk and tumor architecture of glioblastoma. bioRxiv, 2022.2008.2027.505439 (2022). https://doi.org/10.1101/2022.08.27.505439

INFORMAL SEQUENCE LISTING
forward guide:
SEQ ID NO: 1
CACCGAGTTTGTAAAGGACTCCATT;
reverse guide:
SEQ ID NO: 2
AAACAATGGAGTCCTTTACAAACTC;

1. A method for sensitizing glioma cells to immunotherapy in a subject in need thereof, the method comprising administering to the subject an inhibitor of the Sec61 complex.

2. The method of claim 1, wherein the inhibitor reduces at least one of the expression of a Sec61 complex subunit and the activity of a Sec61 complex subunit.

3. The method of claim 2, wherein the Sec61 complex subunit is Sec61G.

4. The method of claim 1, wherein the inhibitor comprises a small-molecular therapeutic agent compound.

5. The method of claim 4, wherein the inhibitor is:

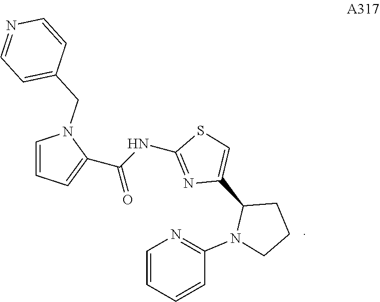 [SMILES: O=C(Nc1nc([C@H]2CCCN2c2ccccn2)cs1)c1cccn1Cc1ccncc1]

6. The method of claim 1, wherein the inhibitor comprises a genome editing system.

7. The method of claim 1, further comprising administering to the subject an immunotherapy for the glioma cells.

8. The method of claim 7, wherein the Sec61 complex inhibitor is administered prior to the immunotherapy.

9. The method of claim 7, wherein the Sec61 complex inhibitor is administered simultaneously with the immunotherapy.

10. The method of claim 1, wherein the glioma cells are glioblastoma cells.

11. The method of claim 1, wherein the subject is a human.

12. A method for inhibiting the Sec61 complex in a glioma cell, the method comprising contacting the cell with a Sec61 complex inhibitor.

13. The method of claim 12, wherein the inhibitor reduces at least one of the expression of a Sec61 complex subunit and the activity of a Sec61 complex subunit.

14. The method of claim 13, wherein the Sec61 complex subunit is Sec61G.

15. The method of claim 12, wherein the inhibitor comprises a small-molecular therapeutic agent.

16. The method of claim 15, wherein the inhibitor is:

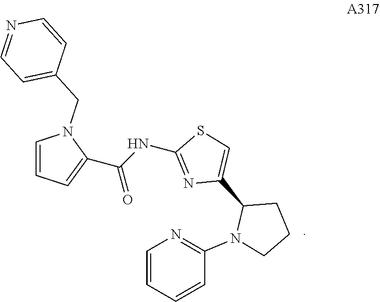 [SMILES: O=C(Nc1nc([C@H]2CCCN2c2ccccn2)cs1)c1cccn1Cc1ccncc1]

17. The method of claim 12, wherein the inhibitor comprises a genome editing system.

18. The method of claim 12, further comprising contacting the cell with an immunotherapeutic agent for the glioma.

19. The method of claim 18, wherein the cell is contacted with the Sec61 complex inhibitor prior to the immunotherapy.

20. The method of claim 18, wherein the cell is contacted with the Sec61 complex inhibitor and the immunotherapy simultaneously.
